(19) United States
(12) Patent Application Publication
(10) Pub. No.: US 2024/0225999 A1
(43) Pub. Date: Jul. 11, 2024

(54) SYSTEMS AND METHODS FOR MICROBOT-MEDIATED THERAPEUTIC DELIVERY

(71) Applicant: BIONAUT LABS LTD., Herzliya (IL)

(72) Inventors: Suehyun CHO, Los Angeles, CA (US); Florent CROS, Los Angeles, CA (US); Alex KISELYOV, San Diego, CA (US); Michael SHPIGELMACHER, Los Angeles, CA (US)

(73) Assignee: BIONAUT LABS LTD., Herzliya (IL)

(21) Appl. No.: 18/562,724

(22) PCT Filed: May 20, 2022
(86) PCT No.: PCT/US22/30213

§ 371 Date: Nov. 20, 2023

Related U.S. Application Data
(60) Provisional application No. 63/191,418, filed on May 21, 2021.
(60) Provisional application No. 63/191,454, filed on May 21, 2021.
(60) Provisional application No. 63/191,515, filed on May 21, 2021.
(60) Provisional application No. 63/191,497, filed on May 21, 2021.
(60) Provisional application No. 63/213,120, filed on Jun. 21, 2021.

(51) Int. Cl. A61K 9/00; A61K 38/19

(52) U.S. Cl. CPC A61K 9/0009; A61K 38/195

(57) ABSTRACT

Provided are systems and miniature devices configured to navigate within a patient to a location therewithin for delivering to induce a localized therapeutic effect. Specifically, the present disclosure provides miniature devices configured to be remotely maneuvered along a path within the patient, where the miniature device is configured to selectively: (i) release a guide substance; (ii) release a payload that induces a physiological response; and/or (iii) produce a microtrauma at locations along the path. Further provided are various methods of treatment using such systems and devices.

### FIELD OF THE INVENTION

The presently disclosed subject matter relates to systems and miniature devices configured to navigate within a patient to induce a localized therapeutic effect, as well as methods of using such systems and devices. Specifically, the present disclosure provides miniature devices configured to be remotely maneuvered within a patient along a path and to release a guide substance or payload and/or produce a microtrauma at locations along the path.

### BACKGROUND OF THE INVENTION

Therapeutics have traditionally been administered to patients in various routes, including, e.g., orally, nasally, intravenously, subcutaneously, intramuscularly. Therapeutic delivery means include, e.g., syringe, pill, capsule, powder, syrup, salve or cream, nebulized spray, aqueous solution, non-aqueous solution, suppository, or transdermal patch.

However, traditional routes and means of delivery suffer from several major drawbacks. For one thing, global administration of a therapeutic throughout an animal's body is not always desirable. Often, due to risk of adverse side effects, it would be preferable to deliver a therapeutic only to a desired target, such as a tumor. Further, some therapeutics are very expensive, and it would be more efficient use of a valuable resource to target the expensive therapeutic only to where it is needed in the patient's body. In many medical applications, it would be useful to use a mobile medical device to move within a living organism. For example, it may be desirable to move an internal device through tissue to a particular desired anatomic location to release a drug.

Additionally, bioavailability and pharmacokinetics may make it very challenging to sustain an ideal effective dosage in the patient, especially proximate to a localized delivery target, e.g., a tumor. The patient's body may metabolize or eliminate a therapeutic, causing the levels of the therapeutic to fluctuate and decline. It would be desirable to have a system and method that would permit on-demand drug release at preferred times in preferred locations in a patient.

Further, traditional therapeutics routes and delivery means require a patient or medical professional to actively follow a dosing regimen. This could include, e.g., taking pills correctly in the right quantity and timing, or correctly dosing and administering an injectable biologic. Some therapeutics may require repeated doctor's visits. There may be various incidental dosing instructions, especially for ingestible drugs, such as avoiding certain foods, such as citrus. These dosing regimens can pose significant problems for patients with limited ability to care for themselves, including, e.g., children and dementia patients. Compliance with dosing regimens can be a burdensome problem for any adult, who may simply forget or may be unable to travel easily to make a doctor's appointment. It would be greatly desirable to have a system and method that could reduce or eliminate the need for people to manage regimen instructions.

Also, many traditional therapeutics administration routes and means are painful or uncomfortable. Reducing, e.g., the frequency and duration of hypodermic injections or intravenous infusions, would offer a substantial improvement to a patient's comfort and quality of life.

The present disclosure provides miniature devices (referred to as microbots, nanobots, or micro-/nano-particles herein) loaded with at least one therapeutic to alleviate these serious shortcomings of traditional therapeutic delivery. Local drug release avoids using larger concentrations, which can limit adverse effects and preserve scarce therapeutics, such as custom-made antibodies. The microbot may release the drug at preferred times, which enhances control and rate of dosing of the therapeutic, and may reduce the need for frequent injections by providing a longer duration of sustained therapy.

### SUMMARY OF THE INVENTION

The present disclosure provides miniature devices (also referred to as microbots, nanobots, or micro-/nano-particles herein) that travel along a specific path in 3-dimensional space within a subject, to deliver a payload or to produce a microtrauma as the device passes through tissue. Further, the miniature device may be directed to a specific locus in the subject for local release of a therapeutic agent.

In one aspect, provided herein are devices, systems, and methods to facilitate delivery of a therapeutic agent to a patient or subject. In some embodiments, provided herein are systems configured to facilitate delivery of a therapeutic agent to a patient, comprising: at least one miniature device having a dimension of 50 nm to 1 cm and configured to be maneuvered along a path within the patient under manipulation by an external non-contact force, said miniature device being configured to selectively release a guide substance at locations along the path; a driving apparatus for creating the external non-contact force to manipulate the miniature device within the patient; and one or more delivery units, each comprising the therapeutic agent and a recognition substance having a high affinity for the guide substance.

In another aspect, provided herein devices, systems, and methods to elicit a physiological response in a patient or subject. In some embodiments, provided herein are systems configured to elicit a physiological response (e.g., an immunological or an inflammatory response) in a patient or subject, comprising: at least one miniature device having a dimension of 50 nm to 1 cm and configured to be maneuvered along a path within the patient under manipulation by an external non-contact force, said miniature device being configured to selectively: release a payload that induces an immunological response at locations along the path; and/or produce a microtrauma at locations along the path; and a driving apparatus for creating said external non-contact force to manipulate the miniature device within the patient.

In some embodiments, the miniature device releases its payload or cargo in response to external stimuli, endogenous stimuli, or both. In some embodiments, the miniature device releases its payload or cargo in response to endogenous stimuli (e.g., temperature, pH, pressure, salinity, enzymes, receptors and/or agonists) within an animal subject. The animal subject may be non-mammalian or mammalian. In preferred embodiments, the animal subject is a human patient.

In some embodiments, the payload or cargo is mounted (e.g., by chemical bonding) on or within the miniature device; or carried in a hollow chamber within the device, or both. In some embodiments, the payload or cargo may be mounted covalently or non-covalently on a solid surface of the miniature device.

In some embodiments, the miniature device releases its payload or cargo in response to an external force. The external force may be mechanomagnetic, electromagnetic, piezoelectric, ultrasound, radiofrequency, optical treatment, or any combination thereof.

In some embodiments, the driving apparatus propels the miniature device from an introduction site, e.g., an injection site, in a subject or patient to a target locus. In some embodiments, the driving apparatus propels the device by means of mechanical (e.g., elastomeric), electromagnetism, ultrasound, radiofrequency, optical, electrical, or a combination thereof.

In some embodiments, the miniature device is propelled from the injection site to the target locus through a biological matrix, a tissue, an organ, circuitry, a vessel, a lumen, or a combination thereof. In some embodiments, the miniature device may be repositioned or removed. In some embodiments, the miniature device is propelled through such a biological matrix, a tissue, an organ, circuitry, a vessel, a lumen, or a combination thereof in order to bore a microtrauma track.

In some embodiments, the miniature device further comprises a coating, wherein the coating renders the payload or cargo inactive (e.g., therapeutically inactive), and wherein removing or disabling the coating activates the payload or cargo. For example, the miniature device may be covered in a film of soluble glycoside that gradually dissolves, exposing the payload or cargo for therapeutic activity and/or release. In an aspect, the coating may be removed or disabled by means of dissolution, dispersion, decomposition, metabolism, pH, redox reaction, or enzymatic machinery present at a locus of therapeutic interest.

The payload or cargo may comprise, by way of example, a biologic, small molecule drug, sugar, lipid, fatty acid, vitamin, mineral, ion or salt, microbe or virus (whether engineered or not), nucleic acid, peptide or protein, or a combination thereof. In some embodiments, the therapeutic cargo may be selected from chemokines, chemokine epitope analogs, chemokine receptor, chemokine receptor epitope analogs, immunoglobulins (including IgG, IgM, IgA, IgD, and IgE), antibodies, antibody constructs, antibody epitopes (including complete sequences, fragments, native epitopes, and engineered epitopes), immunological ligands, immunological cell receptors (including T-cell receptors), interferons, and a combination thereof. The payload or cargo may be selected from the group consisting of: CCL2, CCL3, CCL5, CXCL1, CXCLCXCL5, CXCL6, CXCL8, CXCL9, CXCL10, IFNγ, and a combination thereof.

In some embodiments, the payload or cargo induces a physiological response that modulates, sequesters, collects, immobilizes, deactivates, suppresses, and/or inhibits endogenous immunological molecules or immune cells in a subject. For example, the payload or cargo comprises an immunosuppressant. The physiological response may be optimized to treat an autoimmune condition, such as, psoriasis, rheumatoid arthritis, lupus, multiple sclerosis, celiac disease, Chron's disease, or vasculitis.

In some embodiments, the driving apparatus is configured to manipulate the miniature device to selectively release one or more guide substances and/or recognition substances. In some embodiments the miniature device is configured to release the guide substance according to a predetermined program. In some embodiments, the miniature device is configured to selectively vary the density of the guide substance released along the path. For example, the device is configured to increase the density of the guide substance released as it approaches a target site.

In some embodiments, one of the guide and recognition substances comprises streptavidin, with the other of the substances comprising biotin. In some embodiments, one of the guide and recognition substances comprises chemokine ligand 2 (CCL2), with the other of the substances comprising chemokine receptor type 2 (CCR2). It will be appreciated that the guide and/or recognition substance may comprise a chemical in the sense that it is configured to express it. In some embodiments, the recognition substance is connected to the therapeutic agent via a cleavable linker. The cleavable linker may be a labile chemical bond susceptible to cleavage via an endogenous stimulus. The endogenous stimulus may be selected from a group including an acidic environment, a reduction-oxidation reaction, and an enzyme. The cleavable linker may be a labile chemical bond susceptible to cleavage via an external stimulus. The external stimulus may be selected from an ultrasound signal, an optical signal, and an electrical signal. The recognition substance may be connected to the therapeutic agent via a non-cleavable linker. The therapeutic agent may constitute or comprise the recognition substance. Each delivery unit may be configured to release the therapeutic agent in response to one or more exogenous or endogenous stimuli, for example the recognition substance may comprise a cell. The therapeutic agent and or recognition substance may comprise at least one selected from a group including small molecules, peptides, peptoids, oligonucleotide sequences, nucleic acids, oncolytic viruses, endogenous cells, and engineered cells.

Also provided herein are methods of treating a medical condition in a subject, the method comprising (a) introducing at an introduction site a miniature device described herein; (b) using the driving apparatus to navigate the miniature device from the site introduction site to at least one target treatment locus; and (c) passively or actively causing the payload or cargo to become available for therapeutic activity at the at least one target treatment locus.

In some embodiments, the driving apparatus manipulates the miniature device by means of magnetism. In some embodiments, actively causing therapeutic cargo to become available for therapeutic activity is accomplished by means of a magnetic switch. In other embodiments, actively causing therapeutic cargo to become available for therapeutic activity is accomplished by means of an ultrasound trigger.

In some embodiments, the driving apparatus manipulates or propels the miniature device through a biological matrix, a tissue, an organ, circuitry, a vessel, a lumen, or a combination thereof, in order to bore a microtrauma track.

In some embodiments the animal in need of medical treatment is a mammal. In some embodiments the animal in need of medical treatment is a human patient.

In some embodiments, the medical condition is selected from psoriasis, rheumatoid arthritis, lupus, multiple sclerosis, celiac disease, Crohn's disease, or vasculitis.

In another aspect, provided herein are methods and devices for creating a microtrauma track in a live animal. In some embodiments, the method of creating a microtrauma track in an animal comprises the steps of: (a) introducing a microbot, having a dimension ranging from 50 nm to 1 cm, into the animal's body via an incision, portal, or orifice; and (b) propelling the microbot in a controlled and/or predictable manner through tissue such that the microbot's movement through the tissue creates a microtrauma borehole and/or abrasion in the tissue. In some embodiments, the method of creating a microtrauma track is performed to treat a condition in the animal.

In some embodiments, a driving apparatus drives the microbot to a target locus within the animal. In some embodiments, the driving apparatus propels the microbot by means of: mechanical force, electromagnetism, ultrasound, radiofrequency, optical, electrical or electromotive, or combinations thereof. In some embodiments, the microbot may comprises a propeller. In some embodiments, the tissue through which the microbot is propelled is liver tissue.

In some embodiments, the method also comprises removing the microbot from the subject.

In some embodiments, the microbot may be selected for particular size, surface texture, stiffness, and shape optimal for treating the condition. In some embodiments, the condition may be any of: inflammatory, fibrotic, vascular, neoplastic, metabolic, or any combination thereof. In some embodiments, the target locus may be a tumor.

In some embodiments of the method, the microbot is configured to carry a therapeutic cargo. In some embodiments, the therapeutic cargo is carried in a gated internal chamber inside the microbot; bound covalently or non-covalently to the surface of the microbot; or both.

In some embodiments, the payload or cargo is released in response to endogenous stimuli (within the animal), exogenous stimuli (from outside the animal), or both.

In some embodiments, the payload or cargo is responsive to endogenous repair mechanisms, including: cells, growth factors, cytokines, chemokines, oligonucleotides, platelets, neutrophils, monocytes, macrophages, or combinations thereof. In some embodiments, the payload or cargo is released in response to exogenous stimuli, including: mechanomagnetic, electromagnetic, piezoelectric, ultrasound, radiofrequency, optical, or combinations thereof.

In some embodiments, the microbot gradually releases its payload or cargo as it is propelled through tissue. In some embodiments, the microbot exposes a target locus to the payload or cargo.

In some embodiments of the method, propelling the microbot in a controlled and/or predictable manner through tissue such that the microbot's movement through the tissue creates a microtrauma (e.g., a borehole and/or abrasion in the tissue) is precisely controlled as to produce a desired volume, area, diameter, and/or intensity of microtrauma track in the tissue.

In some embodiments of the method, the payload or cargo comprises any of a cell, biologic, small molecule drug, label, sugar, lipid, fatty acid, vitamin, mineral, ion or salt, microbe, virus, nucleic acid, delivery vector, peptide or protein, or a combination thereof. In some embodiments, the payload or cargo may be an immune response modulator or an inflammation modulator.

In some embodiments, the methods further comprise infusing the microtrauma track with a therapeutic. In some embodiments, infusing the microtrauma track with a therapeutic comprises infusing the microtrauma track with any of a cell, biologic, small molecule drug, label, sugar, lipid, fatty acid, vitamin, mineral, ion or salt, microbe, virus, nucleic acid, vector, peptide or protein, or a combination thereof. In some embodiments, infusing the microtrauma track with a therapeutic comprises infusing it with an immune response modulator or an inflammation modulator.

In some embodiments, one or more method steps are repeated two, three, four, five, six, seven, eight, nine, ten, or more times. In some embodiments, the methods comprise introducing multiple microbots. In some embodiments, the method is repeated using multiple microbots.

In some embodiments, the animal is a mammal, and preferably a human patient.

In another aspect, provided herein are microbots for inducing a microtrauma track in an animal, having a dimension of 50 nm to 1 cm, and optionally carrying a payload or cargo.

In some embodiments, the microbot comprises at least one boring and/or abrasive surface.

In some embodiments, the microbot releases its payload or cargo in response to external stimuli, endogenous stimuli, or both. In some embodiments, the microbot releases its payload or cargo in response to an external force. In some embodiments, the external force is selected from: mechanomagnetic, electromagnetic, piezoelectric, ultrasound, radiofrequency, optical treatment, or a combination thereof. In some embodiments, the microbot releases its payload or cargo in response to endogenous stimuli within the animal.

In some embodiments, the driving apparatus propels the microbot from an introduction site to a target locus within the animal. In some embodiments, the driving apparatus propels the microbot by means of: mechanical, electromagnetism, ultrasound, radiofrequency, optical, electrical, or a combination thereof.

In some embodiments, the payload or cargo is mounted covalently or non-covalently on a solid surface of the microbot.

In some embodiments, the payload or cargo may induce a physiological response that modulates, sequesters, collects, immobilizes, deactivates, suppresses, and/or inhibits endogenous immunological molecules or immune cells in a subject. For example, the payload or cargo may comprise an immunosuppressant.

In some embodiments, the animal is a mammal. Preferably, the mammal is a human patient.

### BRIEF DESCRIPTION OF THE DRAWINGS

FIG. 1 depicts an exemplary schematic drawing of a microbot delivery device. In this exemplary embodiment, the microbot has a magnetic switch that contains a payload designated as “Molecule A” and depicted as contained within a hollow chamber in the microbot delivery device. Molecule A may be released upon engaging the switch.

FIG. 2A depicts an exemplary drawing of a microbot delivery device having a Molecule A covalently attached to the exterior surface of the delivery device.

FIG. 2B depicts an exemplary drawing of a microbot delivery device having a Molecule A non-covalently attached to the exterior surface of the delivery device.

FIG. 3 depicts an exemplary drawing of a microbot delivery device having a coating covering the microbot, which can be removed, e.g., dissolved, permitting controlled, delayed release of Molecule A, which is non-covalently immobilized on a surface of the delivery device.

FIG. 4A depicts a non-limiting exemplary treatment using a microbot of the present disclosure, wherein a microbot (labeled in the drawing as a BIONAUT™) is injected into a patient's cisterna magna to travel to a location in the patient's midbrain.

FIG. 4B depicts another non-limiting exemplary treatment using a microbot (labeled in the drawing as a BIONAUT™). After arriving at a locus of therapeutic interest, e.g., a patient's midbrain, the microbot presents its therapeutic cargo (released from a chamber in the microbot and/or covalently or non-covalently immobilized on the surface of the microbot).

FIG. 5A depicts a schematic drawing of a microbot (labeled in the drawing as a BIONAUT™) to be inserted, as a non-limiting example, in a human patient's liver.

FIG. 5B depicts an exemplary schematic drawing of a microbot (labeled in the drawing as a BIONAUT™) route of travel through a human patient's liver, from the patient's right lobe (left side of the drawing) creating a microtrauma track in the liver as the microbot travels from the insertion point up to the therapeutic target at the anterior right lobe. The microbot may stay at the therapeutic target locus or may be removed.

FIG. 5C depicts an exemplary schematic of the endogenous immune and inflammatory responses, including, e.g., macrophages, platelets, growth factors, elicited by a microtrauma track created by the microbot (labeled in the drawing as a BIONAUT™).

FIG. 5D depicts an exemplary schematic drawing of introduction of a therapeutic payload, e.g., engineered cells, into the microtrauma track. The microtrauma track may provide an access channel to a introduce therapeutic agents to a therapeutic target.

FIG. 6 is a block diagram illustrating a method of using the system of the present disclosure with a guide substance.

FIGS. 7A-7D illustrate photographic bioluminescence data from mice treated as described in the Examples. FIG. 7A illustrates the negative control. FIGS. 7B, 7C, and 7D illustrate three experimental mouse specimens dosed in the right brain hemisphere.

FIGS. 8A-8D illustrate photographic bioluminescence data from mice treated with another embodiment. FIGS. 8A and 8C depict the same individual negative control mouse specimen, while FIGS. 8B and 8D depict the same individual experimental mouse specimen at 11 days of treatment.

FIG. 8B illustrates bioluminescence of a first target in the mouse's left brain hemisphere; FIG. 8D illustrates bioluminescence of a second target in the mouse's right brain hemisphere.

FIG. 9A-9C illustrate photographic bioluminescence data from the same mouse specimen of FIGS. 7B, 7D, at 60 days of treatment. FIG. 9A illustrates bioluminescence of a first target in the mouse's left brain hemisphere; FIG. 9B illustrates bioluminescence of a second target in the mouse's right brain hemisphere; and FIG. 9C illustrates a layered composite image reflecting the bioluminescence data of FIGS. 9A and 9B.

FIG. 10 illustrates a 3-dimensional spatial representation of two different bioluminescence outputs from an experimental mouse treated in the Example.

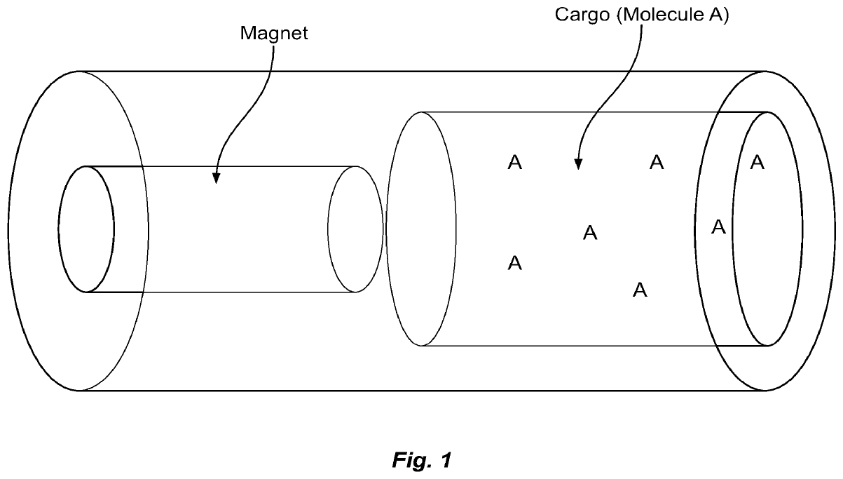

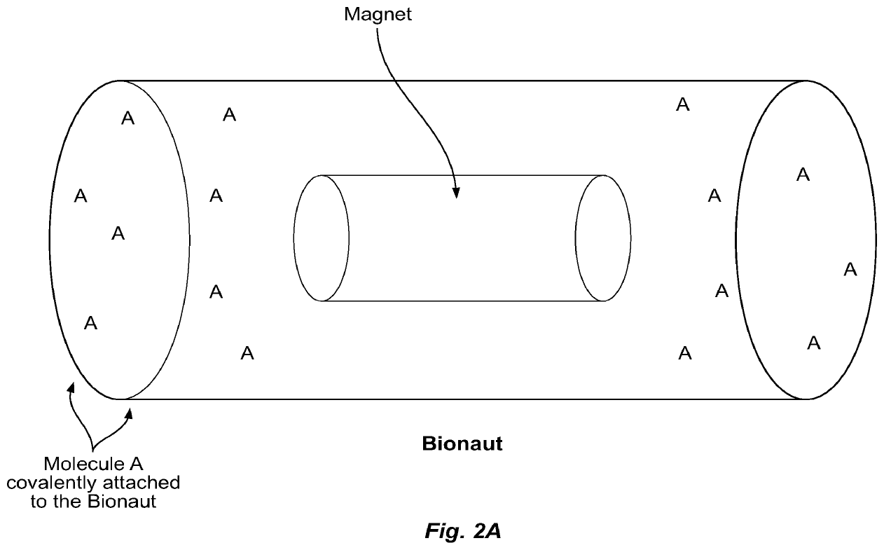

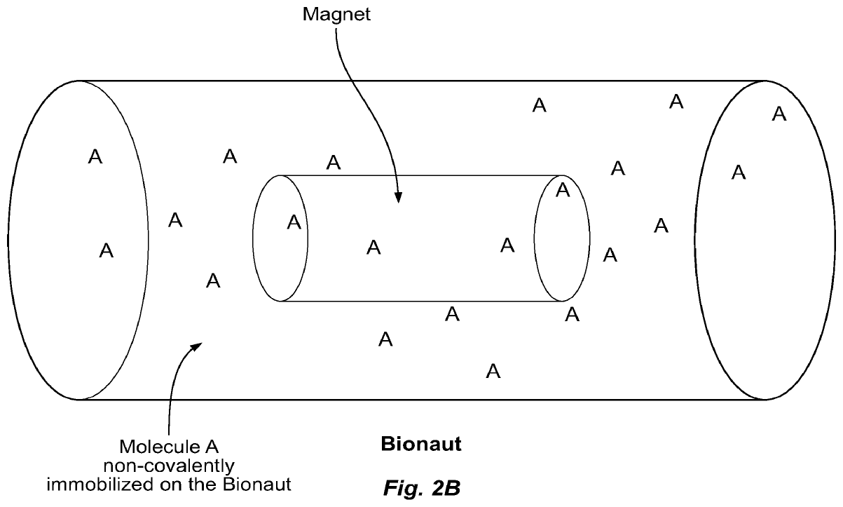

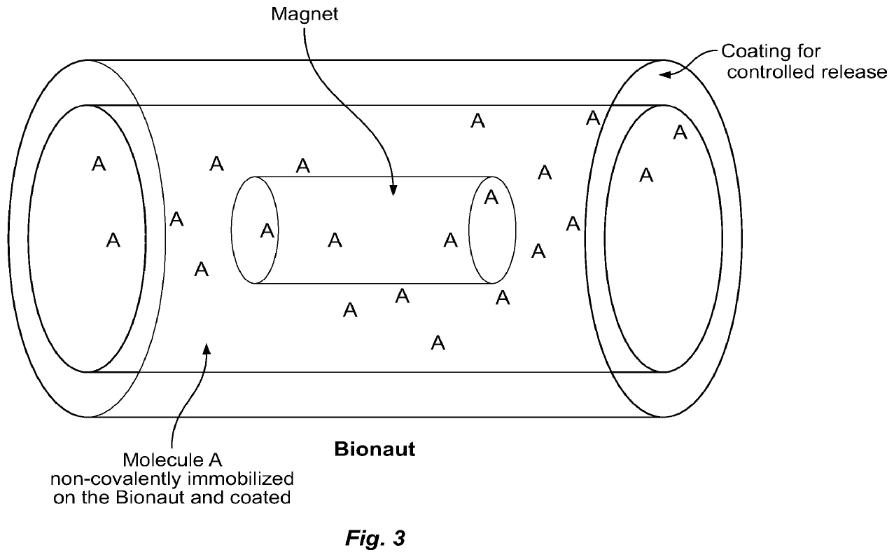

### DETAILED DESCRIPTION OF THE INVENTION

As used here, including the appended claims, the singular forms of words such as “a,” “an,” and “the,” include their corresponding plural references unless context clearly dictates otherwise.

All references cited herein are incorporated by reference to the same extent as if each individual publication, patent application, or patent, was specifically and individually indicated to be incorporated by reference.

In many medical applications, it would be desirable to have a system and method for deploying a motile delivery device to move in a directed way within a living organism. The device may be driven and directed (for example by mechanomagnetic force) through viscous biological medium, from an introduction site to a target locus in the organism. The device could send a therapeutic cargo from an orifice or incision to a localized target such as a tumor. The delivery device could be configured to delay release, or slow-release the therapeutic cargo.

The therapeutic cargo may comprise a payload and/or coating comprising any of a small-molecule drug, biologic, microbe (i.e., bacteria, archaea, and/or single-celled eukaryote), virus, micelles, nutrient, mineral, vitamin, peptide, enzyme, antibody or antibody fragment (whether engineered or natural), nucleic acid (including DNA and/or RNA), carbohydrate, lipid or fatty acid, aqueous solution, non-aqueous solution, nanomaterial, nanoparticle (e.g., immunogold nanoparticle), glue, binder, chemical suture, label (e.g., radiolabel), or any combination thereof.

Thus, in an aspect, the present disclosure provides devices, systems, and methods for delivery of a therapeutic in an animal. In some embodiments, the present disclosure provides a miniature therapeutic delivery device having a dimension of 50 nm to 1 cm, comprising a payload or cargo. A driving apparatus may be configured to drive and direct the miniature device by magnetic means. For example, the driving apparatus may operate according to the disclosures of U.S. Ser. No. 16/609,493 and/or PCT/US2019/041309, which are incorporated by reference in their entireties. The delivery device may be controlled according to the disclosure of U.S. Ser. No. 16/620,748, which is incorporated by reference in its entirety.

In some embodiments, the miniature device may release its payload or cargo in response to external stimuli, endogenous stimuli, or both. In some embodiments the miniature device may release its payload or cargo in response to endogenous stimuli (including, e.g., temperature, pH, pressure, salinity, enzymes, receptors and/or agonists) within an animal subject.

The animal may be non-mammalian or mammalian. Preferably, the animal is human.

In some embodiments, the payload or cargo is mounted (e.g., by chemical bonding) on or within the miniature device; or is carried in a hollow chamber within it, or both. In some embodiments, the cargo or payload is mounted covalently or non-covalently on a solid surface of the miniature device.

In some embodiments, the device releases its payload or cargo in response to an external force. The external force may be selected from mechanomagnetic, electromagnetic, piezoelectric, ultrasound, radiofrequency, optical treatment, or combinations thereof. For example, the cargo or payload is released by exogenous ultrasound stimuli, according to the disclosures of U.S. Ser. No. 17/052,201 and/or PCT/US2018/030949, which are incorporated by reference in their entireties.

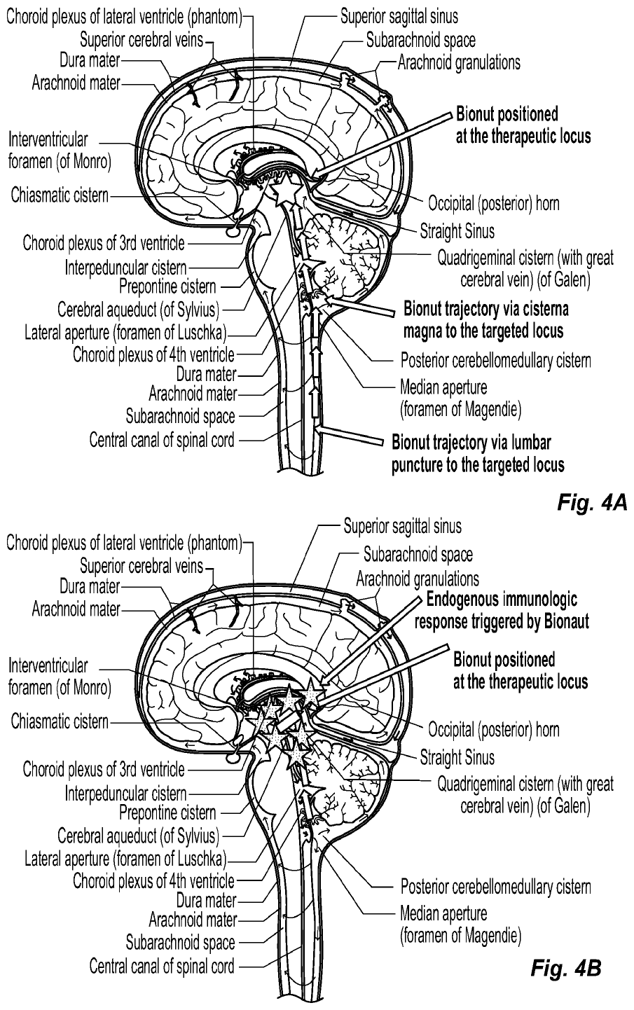

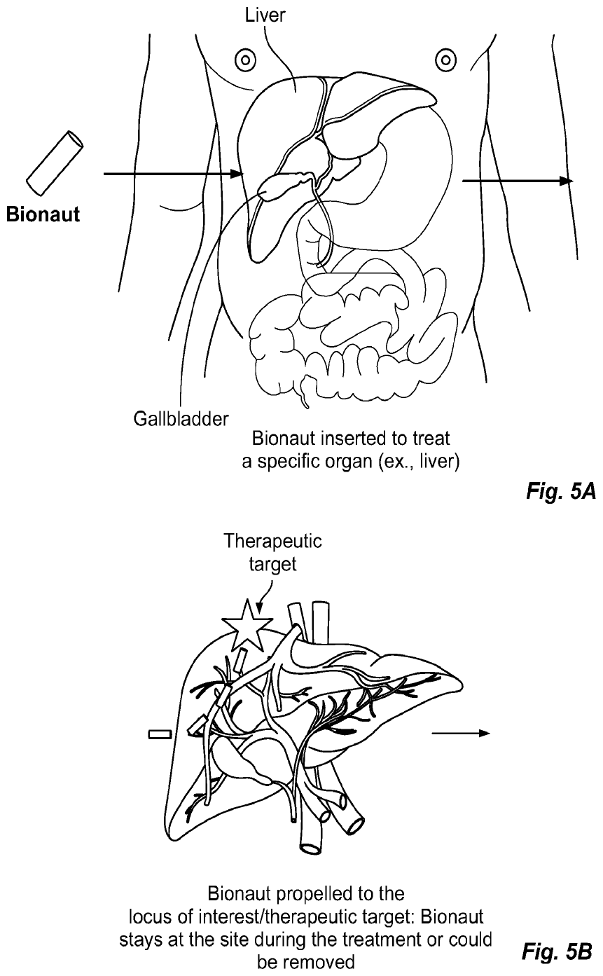

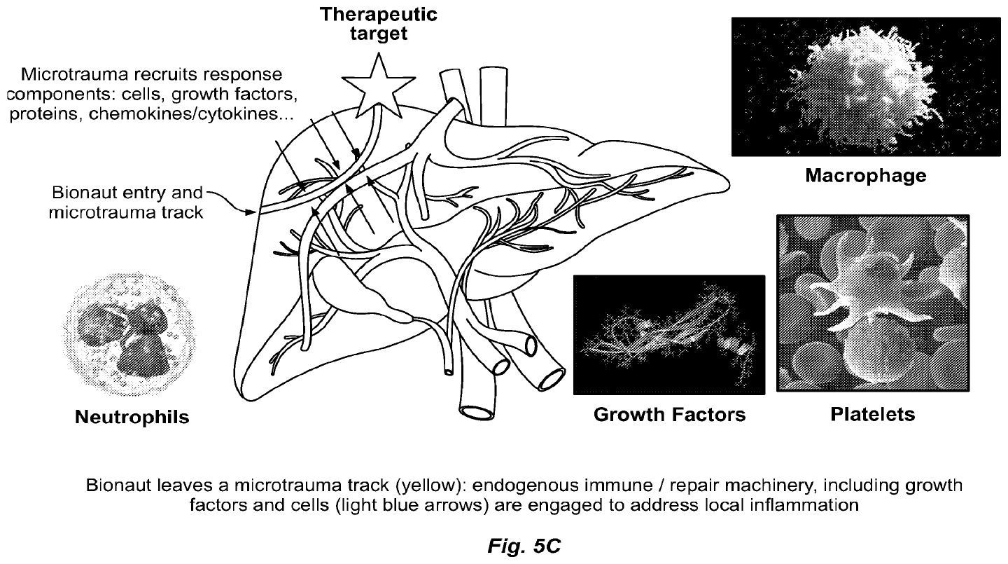

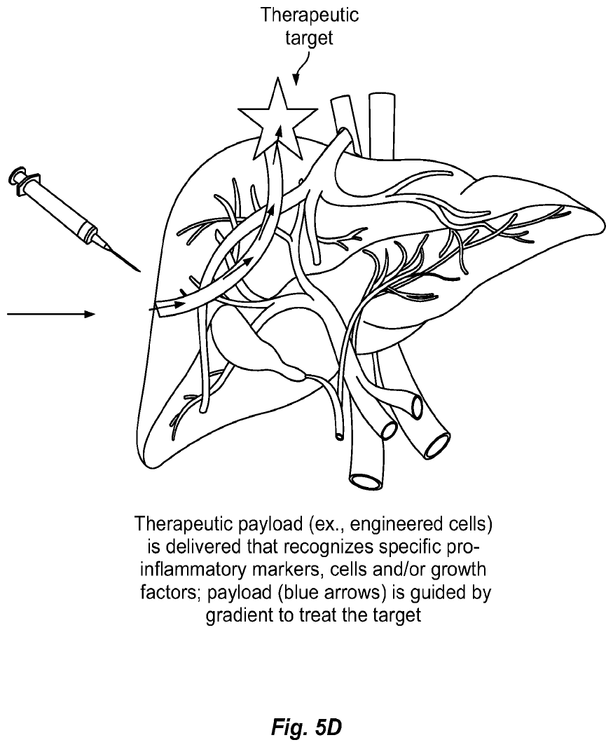

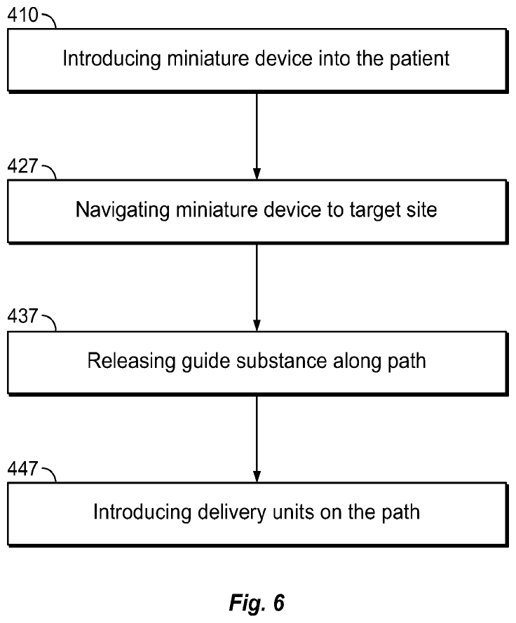

In some embodiments, a driving apparatus drives or propels the miniature device from an introduction site, e.g., an injection site, on an animal subject to a target locus within the subject. For instance, the miniature device is inserted via microcatheter into a patient's lumbar spine, and the driving apparatus propels the device to a locus in the patient's midbrain, as illustrated in FIG. 4A, and presents the therapeutic cargo (by releasing the cargo and/or presenting therapeutic cargo immobilized on a solid surface of the delivery device), as illustrated in FIG. 4B. In another example, the miniature device is inserted into a vessel in a human patient's arm and the driving apparatus propels the device to the patient's liver. In yet another example, the miniature device is inserted into a patient's esophagus and the driving apparatus propels it to a lesion located on the duodenum. In some embodiments, the driving apparatus propels the device by means of electromagnetism, ultrasound, radiofrequency, optical, electrical, or a combination thereof.

In some embodiments, the miniature device is propelled from an injection site to target loci through a biological matrix, a tissue, an organ, circuitry, vessel(s), a lumen, or combinations thereof. The miniature device may be propelled, disclosed in PCT/US2019/059178, which is incorporated by reference in its entirety. In some embodiments, the miniature device is repositioned or removed. More information regarding the deployment and retraction of the devices are found in PCT/US2019/030355, which is incorporated by reference in its entirety.

Further, it is well known that there may be therapeutic benefit to creating carefully planned “microtrauma” in an animal, such as a human patient. Traditional techniques such as acupuncture and cupping have long followed this general concept. Accordingly, a miniature device described herein may be propelled to bore a microtrauma track on a 3-dimensional course through the patient's tissue. Creating a precise, tailored microtrauma track in an animal, e.g., through muscle or connective tissue, or in liver, elicits endogenous therapeutic responses, and also offers a microscopic bore through which additional therapeutics may be infused. The microtrauma track may be bored through a biological matrix, a tissue, an organ, circuitry, vessel(s), a lumen, or a combination thereof.

In some embodiments, the miniature device also comprises a coating, where the coating renders the payload or cargo inactive, and where removing or disabling the coating activates the payload or cargo. For example, the miniature device is covered in a film of soluble glycoside that gradually dissolves, exposing the payload or cargo for therapeutic activity and/or release. The coating may be removed or disabled by means of pH, dissolution, dispersion, decomposition, metabolism, redox reaction, or enzymatic machinery present at a locus of therapeutic interest.

The therapeutic cargo may comprise, by way of example, a biologic, small molecule drug, sugar, lipid, fatty acid, vitamin, mineral, ion or salt, microbe or virus (whether engineered or not), nucleic acid, peptide or protein, or any combination thereof. In some embodiments, the therapeutic cargo is selected from chemokines, chemokine epitope analogs, chemokine receptor, chemokine receptor epitope analogs, immunoglobulins (including IgG, IgM, IgA, IgD, and IgE), antibodies, antibody constructs, antibody epitopes (including complete sequences, fragments, native epitopes, and engineered epitopes), immunological ligands, immunological cell receptors (including T-cell receptors), interferons, and combinations thereof. The therapeutic cargo may be selected from the group consisting of: CCL2, CCL3, CCL5, CXCL1, CXCLCXCL5, CXCL6, CXCL8, CXCL9, CXCL10, IFNγ, and combinations thereof.

In some embodiments, the payload or cargo induces a physiological response that modulates, sequesters, collects, immobilizes, deactivates, suppresses, and/or inhibits endogenous immunological molecules or immune cells in a subject. For example, the payload or cargo comprises an immunosuppressant. Immunosuppressants may be small molecule drugs and/or biologics (e.g., abatacept, adalimumab, anakinra, basiliximab, certolizumab, daclizumab, etanercept, golimumab, infliximab, ixekizumab, natalizumab, rituximab, secukinumab, tocilizumab, ustekinumab, vedolizumab, etc.) and may include, without limitation, calcineurin inhibitors (cyclosporine, tacrolimus, etc.), corticosteroids (e.g., budesonide, prednisolone, prednisone, etc.), inosine monophosphate dehydrogenase (IMDH) inhibitors (azathioprine, leflunomide, mycophenolate, etc.), Janus kinase inhibitors (tofacitinib, etc.), mTOR inhibitors (everolimus, sirolimus, etc.), and others.

The physiological response may be optimized to treat an autoimmune condition, such as, psoriasis, rheumatoid arthritis, lupus, multiple sclerosis, celiac disease, Crohn's diseases, or vasculitis.

In some embodiments, the driving apparatus is configured to manipulate the miniature device to selectively release one or more guide substances and/or recognition substances. In some embodiments, the miniature device is configured to release the guide substance according to a predetermined program. In some embodiments, the miniature device is configured to selectively vary the density of the guide substance released along the path. The device is configured to increase the density of the guide substance released as it approaches a target site.

In some embodiments, one of the guide and recognition substances comprises streptavidin, with the other of the substances comprising biotin. In some embodiments, one of the guide and recognition substances comprises chemokine ligand 2 (CCL2), with the other of the substances comprising chemokine receptor type 2 (CCR2). It will be appreciated that the guide and/or recognition substance may comprise a chemical in the sense that it is configured to express it. In some embodiments, the recognition substance is connected to the therapeutic agent via a cleavable linker. The cleavable linker may be a labile chemical bond susceptible to cleavage via an endogenous stimulus. The endogenous stimulus may be selected from an acidic environment, a reduction-oxidation reaction, and an enzyme. The cleavable linker may be a labile chemical bond susceptible to cleavage via an external stimulus. The external stimulus may be selected from an ultrasound signal, an optical signal, and an electrical signal. The recognition substance may be connected to the therapeutic agent via a non-cleavable linker. The therapeutic agent may constitute or comprise the recognition substance. Each delivery unit may be configured to release the therapeutic agent in response to one or more exogenous or endogenous stimuli, for example, the recognition substance may comprise a cell. The therapeutic agent may comprise at least one selected from a group including small molecules, peptides, peptoids, oligonucleotide sequences, nucleic acids, oncolytic viruses, endogenous cells, and engineered cells. The recognition substance may be selected from a group including a molecule and a cell.

Also provided herein are methods of treating a medical condition in an animal in need thereof, the method comprising (a) introducing at an introduction site a miniature device described herein into the animal; (b) using the driving apparatus to manipulate or propel the miniature device from the introduction site to at least one target treatment locus; and (c) passively or actively causing a therapeutic cargo to become available for therapeutic activity at the at least one target treatment locus.

In one embodiment, the driving apparatus manipulates the miniature device magnetically. In some embodiments, actively causing therapeutic cargo to become available for therapeutic activity is achieved using a magnetic switch. In other embodiments, actively causing therapeutic cargo to become available for therapeutic activity is achieved using an ultrasound trigger.

In some embodiments the animal in need of medical treatment is a mammal. In some embodiments the animal in need of medical treatment is a human patient.

The methods of treating a medical condition may be used to treat psoriasis, rheumatoid arthritis, lupus, multiple sclerosis, celiac disease, Crohn's disease, or vasculitis.

In any aspect or embodiment, the miniature devices may be packed and delivered in the form of a pill, capsule, cream, salve, syrup, dermal patch, suppository, intravenous drip, aqueous solution, non-aqueous solution, or any combination thereof. In any aspect or embodiment, the miniature device may be prepackaged in a container, pack, or syringe. In any aspect or embodiment, the selected composition may further comprise instructions for administration. In any aspect or embodiment, the miniature device may be administered to an animal by ingestion, intravenous injection, peritoneal injection, muscular injection, nasally, orally, ocularly, rectally, or any combination thereof. In any aspect or embodiment, the therapeutic cargo is packed into the delivery device in pharmaceutically effective amounts and/or concentrations.

Further, devices described herein may include creating controlled “microtrauma” that evokes a localized and predictable inflammatory and immunological response along a “track” bored or otherwise left in the tissue. A designated payload or cargo may be carried by the device or infused into the track. The cargo may be a therapeutic or may be a label, allowing a physician to locate and identify the site of the microtrauma track.

Meeting these needs, provided herein are methods, systems and devices for creating a microtrauma track in an animal. In some embodiments, a method of creating a microtrauma track in an animal comprises: (a) introducing a microbot, having a dimension ranging from 50 nm to 1 cm, into the animal via an incision, portal, or orifice; and (b) propelling the microbot in a controlled and/or predictable manner through tissue such that its movement through the tissue creates a microtrauma borehole in the tissue. For example, a physician may create an incision in a human patient's dermis and introduce a microbot, which then bores a microtrauma track through the patient's muscle. The microbot may be navigated to bore a microtrauma track through a biological matrix, a tissue, an organ, musculature, circuitry, vessel(s), a lumen, or any combination thereof.

The region proximate to the microtrauma track triggers endogenous repair mechanisms including, but not limited to, cells, growth factors, cytokines, chemokines, oligonucleotides (including miRNAs/RNAi), etc. The region proximate to the microtrauma track will also invite immunologic and/or inflammatory machinery including, e.g., platelets, neutrophils, monocytes, macrophages, growth factors (e.g., VEGF, PDGF), etc.

In some embodiments, creating a microtrauma track may be done to treat a condition.

In some embodiments, the microbot may be selected for particular size, surface texture, stiffness, and shape optimal for treating a condition. For instance, if a human patient requires a 50 m-diameter microtrauma track created in her hamstring muscle, a suitable microbot of 50 m size may be selected. The anterior surface (i.e., on the front-facing/forward-facing side defined by the direction of movement) of the microbot may be textured and/or fitted with a cutting shape or apparatus, such as one or more blades, to facilitate cutting through stiff or viscous biological tissue and/or matrix. Such surfaces, textures, cutting apparatus, etc., may be configured to generate a microtrauma track as the delivery device passes through a subject's tissues.

In some embodiments, the condition may be: inflammatory, fibrotic, vascular, neoplastic, metabolic, or a combination thereof. In some embodiments, the target locus may be a tumor.

In some embodiments, the microbot is configured to carry a therapeutic cargo. In some embodiments, the therapeutic cargo is carried in a gated internal chamber inside the microbot; bound covalently or non-covalently to the surface of the microbot; or both.

The therapeutic cargo may be a label, such as, fluorescence labels, dyes, radiolabels, linked to e.g., antibodies, antibody constructs, antibody epitopes (including complete sequences, fragments, native epitopes, and engineered epitopes), ligands, hybridizable oligonucleotides (including, e.g., dsDNA, ssDNA, dsRNA, ssRNA, cDNA), cells and other labels known in the art. The labels may be used to identify the site of the microtrauma track and locate a therapy locus.

In some embodiments, the therapeutic cargo is released in response to endogenous stimuli (within the animal), exogenous stimuli (from outside the animal), or both. In some embodiments, the therapeutic cargo is responsive to endogenous repair mechanisms, including: cells, growth factors, cytokines, chemokines, oligonucleotides, platelets, neutrophils, monocytes, macrophages, or combinations thereof. In some embodiments, the therapeutic cargo is responsive to exogenous stimuli, including: mechanomagnetic, electromagnetic, piezoelectric, ultrasound, radiofrequency, optical treatment, or combinations thereof.

In some embodiments, the microbot gradually releases its payload or cargo while it is propelled through the animal's tissue. In some embodiments, the microbot may expose a target locus in the animal to its payload or cargo.

In some embodiments, propelling the microbot in a controlled and/or predictable manner through tissue to create a microtrauma (e.g., a borehole and/or abrasion) is precisely controlled to produce a desired volume, area, diameter, and/or intensity of microtrauma track in the tissue.

In some embodiments, the therapeutic cargo comprises a cell, biologic, small molecule drug, sugar, lipid, fatty acid, vitamin, mineral, ion or salt, microbe, virus, nucleic acid, vector, peptide or protein, or a combination thereof. These include, peptoids, encoding DNA sequences, RNAi, siRNA, miRNA, shRNA, and AAV-based therapeutics. In some embodiments, the therapeutic cargo modulates an immune response or inflammation. The therapeutic cargo may comprise immunologic and/or inflammatory molecules to enhance an endogenous immunologic and/or inflammatory response along a region proximate to the microtrauma track.

In some embodiments, the method further comprises infusing the microtrauma track with a therapeutic. In some embodiments, infusing the microtrauma track with a therapeutic comprises infusing it with any of a cell, biologic, small molecule drug, label, sugar, lipid, fatty acid, vitamin, mineral, ion or salt, microbe, virus, nucleic acid, vector, peptide or protein, or a combination thereof. In some embodiments, infusing the microtrauma track with a therapeutic comprises infusing the microtrauma track with a modulator of an immune response or of inflammation.

In some embodiments, one or more steps of the method are repeated one, two, three, four, five, six, seven, eight, nine, ten, or more times. In some embodiments, the method comprises introducing multiple microbots. In some embodiments, the method comprises performing multiple (i.e., more than one) iterations of the steps concurrently or simultaneously. In some embodiments, the method is repeated using multiple microbots.

In some embodiments, the animal is a mammal, preferably a human patient.

In another aspect, provided herein are microbots for inducing a microtrauma track in an animal, having a dimension of 50 nm to 1 cm, and optionally carrying a therapeutic cargo.

In some embodiments, the microbot comprises at least one boring and/or abrasive surface. The boring and/or abrasive surface may be on an anterior surface (i.e., on the front-facing/forward-facing side defined by the direction of movement) of the microbot, and may be textured and/or fitted with a cutting shape or apparatus, such as one or more blades, to facilitate cutting through stiff or viscous biological tissue and/or matrix.

In some embodiments, the microbot releases its payload or cargo in response to external stimuli, endogenous stimuli, or both. In some embodiments, the microbot releases its payload or cargo in response to an external force, the external force selected from: mechanomagnetic, electromagnetic, piezoelectric, ultrasound, radiofrequency, optical treatment, or a combination thereof. In some embodiments, the microbot releases its payload or cargo in response to endogenous stimuli within the animal.

In some embodiments, the driving apparatus propels the microbot from an introduction site on the subject to a target locus within the subject. In some embodiments, the driving apparatus propels the microbot by means of: mechanical, electromagnetism, ultrasound, radiofrequency, optical, electrical, or any combination thereof.

In some embodiments, the therapeutic cargo is mounted covalently or non-covalently on a solid surface of the microbot.

In some embodiments, the payload or cargo induces a physiological response that modulates, sequesters, collects, immobilizes, deactivates, suppresses, and/or inhibits endogenous immunological molecules or immune cells in a subject. For example, the payload or cargo may comprise an immunosuppressant.

In some embodiments, the therapeutic cargo may comprise a payload and/or coating comprising a cell, small-molecule drug, biologic, microbe (i.e., bacteria, archaea, and/or single-celled eukaryote), virus, micelles, nutrient, mineral, vitamin, peptide, enzyme, antibody or antibody fragment (engineered or natural), nucleic acid (including DNA and RNA), carbohydrate, lipid or fatty acid, aqueous or non-aqueous solution, nanomaterial, nanoparticle (e.g., immunogold nanoparticle), glue, binder, chemical suture, label (e.g., radiolabel), or a combination thereof.

In some embodiments, the animal is a mammal. Preferably, the mammal is a human.

### Examples

A demonstration of the system of the present disclosure was performed in mouse, wherein the miniature delivery device delivered a payload recombinant adeno-associated virus (AAV) construct vector to a locus in either right hemisphere or left hemisphere brain.

Animals were grouped into negative control (no vector) and treated. In a first experiment, a firefly luciferase vector AAV1-CAG-LUCR was placed on a miniature delivery devices. The devices were suspended in solution at a density of about 1×10⁹ vp/μL and then injected into the mouse. A user steered the device to the animals' right hemispheres.

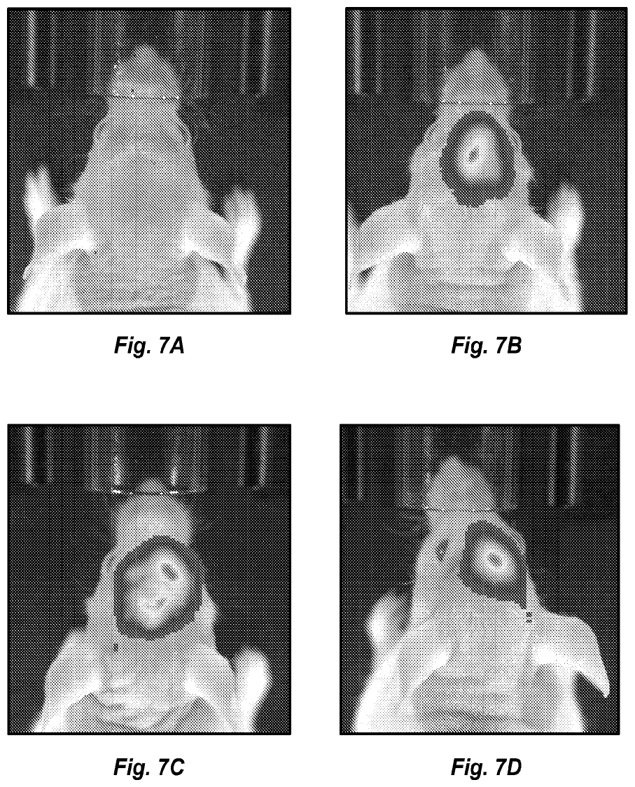

Mice were dosed daily with 1 μL or less of AAV-CAG-Luc(f), and expression levels were mapped by bioluminescence at day 7, day 11, day 20, and day 60 posttreatment. FIG. 7A depicts a negative control mouse having no bioluminescence. FIGS. 7B-7D depict bioluminescence local to right hemisphere at day 7 posttreatment, indicating expression of firefly luciferase.

In another experiment, mice were injected with two separate constructs having two different luciferase homologs to demonstrate localization efficacy. Aimed for the brain right hemisphere were miniature devices loaded with a payload of AAV1-LUC(Renilla) and aimed for the brain left hemisphere were miniature devices loaded with a payload of AAV1-LUCR(Firefly). The devices were suspended in solution at a density of about 1×10⁹ vp/μL and then injected into the mouse. A user steered the device loaded with AAV1-LUC(Renilla) to the right hemisphere, and then steered the device loaded with AAV1-LUCR(Firefly) to the left hemisphere.

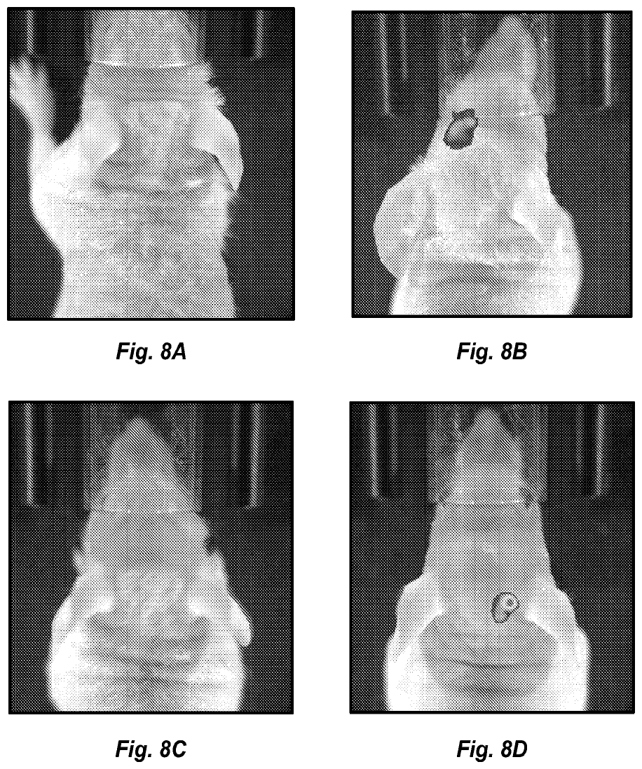

The mice were dosed daily with 1 μL or less of AAV-CAG-Luc(f), and expression levels were mapped by bioluminescence at day 7, day 11, day 20, and day 60 posttreatment. FIGS. 8A and 8C show the same negative control animal, imaged for Renilla luciferase (FIG. 8A) and firefly luciferase (FIG. 8C) at day 11 posttreatment. FIG. 8B shows imaging for Renilla luciferin luminescence, which can be seen localized around the left side of the head; FIG. 8D shows imaging for firefly luciferin luminescence, which can be seen localized around the head's right side.

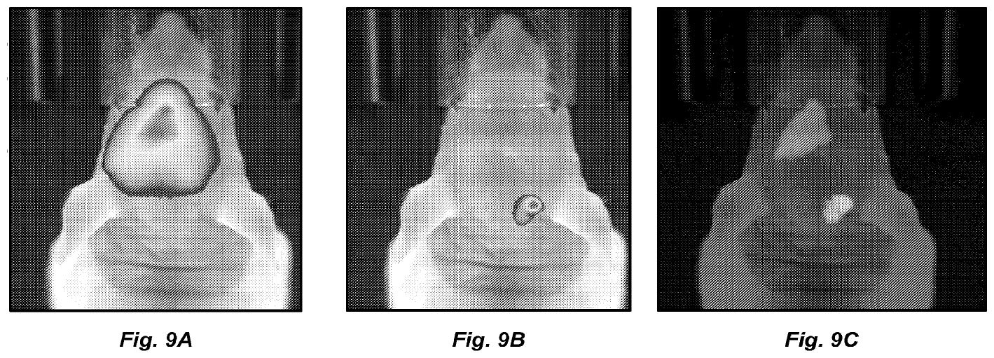

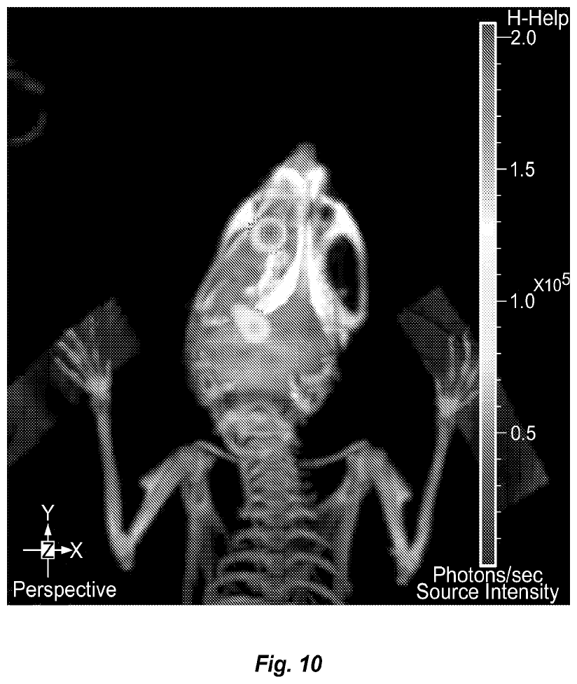

FIGS. 9A-9C depict the same individual specimen as in FIGS. 8B, 8D, imaged in day 60 within 1 hour of Renilla luciferin and firefly luciferin injection. FIG. 9C is an overlay composite showing respective localizations. FIG. 10 shows the 3-dimensional spatial separation of Renilla luminescence and firefly luminescence.

The various embodiments described herein can be combined to provide further embodiments. All U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A system configured to facilitate delivery of a therapeutic agent to a patient, the system comprising:
at least one miniature device configured to be maneuvered along a path within the patient under manipulation by an external non-contact force, said miniature device being configured to selectively release a guide substance at locations along the path;
a driving apparatus for creating said external non-contact force to manipulate the miniature device within the patient; and
one or more delivery units, each comprising the therapeutic agent and a recognition substance having a high affinity for the guide substance.

2. The system according to claim 1, wherein said apparatus is configured to manipulate the miniature device to selectively release the guide substance.

3. The system according to claim 1, wherein said miniature device is configured to release said guide substance according to a predetermined program.

4. The system according to claim 1, wherein one of said guide and recognition substances comprises streptavidin, and the other of the guide and recognition substances comprises biotin.

5. The system according to claim 1, wherein one of said guide and recognition substances comprises chemokine ligand 2 (CCL2), and the other of the guide and recognition substances comprises chemokine receptor type 2 (CCR2).

6. The system according to claim 1, wherein the recognition substance is connected to the therapeutic agent via a cleavable linker.

7. The system according to claim 6, wherein said cleavable linker is a labile chemical bond susceptible to cleavage via an endogenous stimulus.

8. The system according to claim 7, wherein the endogenous stimulus is selected from an acidic environment, a reduction-oxidation reaction, or an enzyme.

9. The system according to claim 7, wherein said cleavable linker is a labile chemical bond susceptible to cleavage via an external stimulus.

10. The system according to claim 10, wherein said external stimulus is selected from an ultrasound signal, an optical signal, and an electrical signal.

11. The system according to claim 1, wherein said delivery units are configured to release the therapeutic agent in response to one or more exogenous or endogenous stimuli.

12. The system according to claim 1, wherein said therapeutic agent is selected from a small molecule, a peptide, a peptoid, an oligonucleotide sequence, a nucleic acid, an oncolytic virus, an endogenous cell, and/or an engineered cell.

13. The system according to claim 1, wherein the recognition substance is selected from a small molecule, a peptide, a peptoid, an oligonucleotide sequence, a nucleic acid, an oncolytic virus, an endogenous cell, and/or an engineered cell.

14. A system configured to elicit a physiological response in a patient, the system comprising:
at least one miniature device configured to be maneuvered along a path within the patient under manipulation by an external non-contact force, said miniature device being configured to selectively:
release a payload that induces an immunological response at locations along the path; and/or
produce a microtrauma at locations along the path; and
a driving apparatus for creating said external non-contact force to manipulate the miniature device within the patient.

15. The system according to claim 14, wherein the payload may modulate, sequester, collect, immobilize, deactivate, suppress, and/or inhibit endogenous immunological molecules or immune cells in the patient.

16. The system according to claim 14, wherein the payload comprises an immunosuppressant.

17. The system according to claim 14, wherein the miniature device is optimized for the treatment of an autoimmune condition.

18. The system according to claim 14, wherein the autoimmune condition is selected from psoriasis, rheumatoid arthritis, lupus, multiple sclerosis, celiac disease, Crohn's disease, or vasculitis.

19. The system according to claim 14, wherein the payload is selected from the group consisting of: chemokines, chemokine epitope analogs, chemokine receptor, chemokine receptor epitope analogs, immunoglobulins (including IgG, IgM, IgA, IgD, and IgE), antibodies, antibody constructs, antibody epitopes (including complete sequences, fragments, native epitopes, and engineered epitopes), immunological ligands, immunological cell receptors (including T-cell receptors), interferons, and any combination thereof.

20. The system according to claim 14, wherein the payload is selected from the group consisting of: CCL2, CCL3, CCL5, CXCL1, CXCLCXCL5, CXCL6, CXCL8, CXCL9, CXCL10, IFNγ, and any combination thereof.

21. The system according to claim 14, wherein the miniature device releases the payload in response to an external force, the external force selected from: mechanomagnetic, electromagnetic, piezoelectric, ultrasound, radiofrequency, optical treatment, or any combination thereof.

22. The system according to claim 14, wherein the miniature device releases the payload in response to endogenous stimuli within the subject.

23. The system according to claim 14, wherein said miniature device further comprises a coating configured to at least partially dissipate under one or more predetermined conditions, thereby releasing the payload.

24. The system according to claim 23, wherein the coating is removed or disabled by means of dissolution, dispersion, decomposition, metabolism, pH, RedOx reaction, or enzymatic machinery present at a locus of therapeutic interest.

25. The system according to claim 14, wherein said payload is carried on an exterior surface of the miniature device.

26. The system according to claim 14, wherein the miniature device comprises at least one boring and/or abrasive surface to produce the microtrauma as the miniature device is manipulated within the patient.

27. A method for delivering a therapeutic agent in a patient, the method comprising:
providing a system according to any one of claims 1 through 15;
introducing the miniature device of the system into the patient at an injection site;
operating the driving apparatus of the system to navigate the miniature device along a path to a target site;
releasing said guide substance at locations along said path; and
introducing said delivery units of the system into the patient;
wherein the delivery units autonomously travel along the path toward the target site, guided by interactions between the recognition substance and the guide substance disposed along the path.

28. The method according to claim 27, wherein introducing said delivery units comprises systemically administering said delivery units.

29. The method according to claim 27, wherein the density of the guide substance released varies along said path.

30. The method according to claim 29, wherein the density of the guide substance released increases as it approaches a target site.

31. A method for inducing a physiological response within a patient, the method comprising:
providing a system according to any one of claims 16 through 26;
introducing the miniature device of the system into the patient at an injection site;
operating the driving apparatus of the system to navigate the miniature device along a path to a target site;
releasing the payload and/or producing the microtrauma at locations along said path to induce a physiological response at said locations.

32. The method according to claim 31, wherein the physiological response is an immunological and/or an inflammatory response.

33. The method according to claim 31, wherein the physiological response itself is therapeutic.

34. The method according to claim 31, wherein the intensity of the physiological response varies along said path.

35. The method according to claim 34, wherein the intensity of the physiological response increases as it approaches a target site.

36. A method for delivering a therapeutic agent in a patient, the method comprising:
providing a system according to claim 26;
introducing the miniature device of the system into the patient at an injection site;
operating the driving apparatus of the system to navigate the miniature device along a path to a target site;
producing a microtrauma track at locations along said path; and
introducing one or more delivery units into the patient;
wherein said delivery units comprise the therapeutic agent and have an affinity for the microtrauma track.

37. The method according to claim 36, wherein introducing said delivery units comprises systemically administering said delivery units.

38. The method according to claim 36, wherein the delivery unit is responsive to endogenous repair mechanisms

39. The method according to claim 38, wherein the delivery units comprise cells, growth factors, cytokines, chemokines, oligonucleotides, platelets, neutrophils, monocytes, macrophages, or a combination thereof.
